(19) United States
(12) Patent Application Publication
(10) Pub. No.: US 2023/0324412 A1
(43) Pub. Date: Oct. 12, 2023

(54) METHODS AND COMPOSITIONS TO DETERMINE THE QUALITY OF RED BLOOD CELL UNITS

(71) Applicants: INSERM (INSTITUT NATIONAL DE LA SANTÉ ET DE LA RECHERCHE MÉDICALE), Paris (FR); ETABLISSEMENT FRANÇAIS DU SANG (EFS), La Plaine Saint Denis (FR); UNIVERSITÉ PARIS-EST CRÉTEIL VAL DE MARNE, Creteil (FR)

(72) Inventors: Véronique BAUDIN-CREUZA, Creteil (FR); Philippe CHADEBECH, Creteil (FR); Corinne VASSEUR, Creteil (FR); France PIRENNE, Creteil (FR)

(21) Appl. No.: 17/995,452

(22) PCT Filed: Apr. 8, 2021
(86) PCT No.: PCT/EP2021/059189

§ 371 Date: Oct. 4, 2022

(30) Foreign Application Priority Data
Apr. 9, 2020 (EP) 20315136.0

(51) Int. Cl. G01N 33/72; G01N 33/68

(52) U.S. Cl. CPC G01N 33/721; G01N 33/6848

(57) ABSTRACT

The present invention relates to a method for determining the quality of haemoglobin (Hb) during the storage period of red blood cell (RBC) units comprising a step of detecting soluble alpha-haemoglobin (α-Hb) pool in RBC lysates and concluding that the presence of α-Hb pool indicates a conservation of quality of Hb during the storage RBCs. Inventors have determined the impact of RBC units aging on the quality of Hb and on the soluble α-Hb pool level in RBCs. For this purpose, 21 RBC units were collected, stored at +4 to 6° C. and samples were taken at two different storage times (D3-D8 and D38-D42) to evaluate spectral characteristics of Hb and soluble α-Hb pool in RBCs. Two additional samples were collected from 16 RBC units, at intermediate time points during storage (D13-D17 and D24-D29; n=16). The α-Hb dosing assay uses the specific character of the interaction between the α-Hb and the AHSP, the α chaperone, to trap the α-Hb present in the RBC lysates of RBC units. They also investigated the effect of a short cryopreservation period at −80° C. for 15 days on the α-Hb pool for 4 different RBC units.

### FIELD OF THE INVENTION

The invention is in the field of haematology. More particularly, the invention relates to methods and compositions to determine the quality of red blood cells during the storage period.

### BACKGROUND OF THE INVENTION

One of the essential objectives of red blood cell (RBC) transfusion is to increase the concentration of haemoglobin (Hb) to improve oxygen delivery to the tissues. During their lifespan in circulation, RBCs undergo morphological and physiochemical modifications.^{1, 2} An increased rigidity of RBCs, the expression of neoantigenic domains or externalization of anionic phospholipids are well documented.^{3, 4} The anion exchanger protein or Banda, also aggregates on the surface of the RBC membranes and becomes the target of senescence autoantibodies.⁵ All the characteristics affecting the RBCs in circulation are also found during the aging of the stored RBCs in blood bags and named in that case “storage lesions”. Microparticle formation are also observed.^{6, 7}

The optimal conservation of stored RBCs in units up to 42 days in blood banks is based on the use of preservative solutions, generally the saline adenine-glucose-mannitol solution (SAGM).^{8, 9} Over this period of storage, RBC units are supposed to keep their optimal ability for deformability to easily circulate into the microvascular capillary network and their capacity to off-load oxygen to the tissues. Despite this, RBCs progressively accumulate irreversible lesions in blood bags,^{6, 7, 10, 11} altering their half-life in circulation when transfused.^{12, 13} However we previously showed that the conventional storage near +4° C. of RBC units over 42 days did not affect the appearance of specific senescence markers detected on RBCs such as calcium influx and detection of reactive oxygen species in cytosol, decrease in size and membrane externalization of phosphatidylserine.¹⁴

RBC is a cellular entity particularly subject to oxidative stress and alterations by free radicals,¹⁵ and Hb is the first candidate for this stress. The human adult Hb (Hb A α₂⊖₂) is constituted of two α-chains and two β-chains, each subunit is associated with the heme molecule having at its center an iron atom in ferrous state, the reversible site of oxygen. Hb can exist in both oxygenated and deoxygenated forms, each with its own characteristic absorbance spectrum. However there are few spectral differences between oxygenated tetrameric Hb and the isolated oxygenated β and α0 subunits¹⁶. Only α₂⊖₂ tetramers can deliver oxygen efficiently, even though the individual β-Hb and α-Hb subunits have a very strong affinity for oxygen and therefore do not efficiently deliver oxygen to tissues. In a previous study, we demonstrated the presence of a soluble α-Hb pool in the RBC lysates of healthy subjects.¹⁷ These results are consistent with those of literature that show a slight excess of α-subunit synthesis described in normal RBCs.¹⁸ Until today, no data on the presence of isolated subunits has been explored, either at the level of the RBCs during their conservation, or in frozen and thawed RBC units, frequently transfused to sickle cell disease patients.

### SUMMARY OF THE INVENTION

The invention relates to a method for determining the quality of haemoglobin (Hb) during the storage period of red blood cell (RBC) units comprising a step of measuring the value of soluble alpha-haemoglobin (α-Hb) pool in RBC units and concluding a conservation of quality of Hb during the storage of RBCs when the value of α-Hb remains stable and does not increase significantly. In particular, the invention is defined by claims.

### DETAILED DESCRIPTION OF THE INVENTION

Inventors have determined the impact of red blood cell (RBC) units aging on the quality of Hb by the measurement of the value of soluble α-Hb pool level in RBCs.

For this purpose, blood from twenty-one healthy adult donors was collected into sterile blood bags containing citrate phosphate dextrose (CPD) as an anticoagulant, at the Etablissement Français du Sang (EFS). These fresh samples were maintained at a temperature of +18° C. to +24° C. for 2 to 24 hours before processing, in accordance with European guidelines¹⁹, at EFS Preparation Unit (Rungis, France). RBCs were isolated by plasma removal and leukoreduction at room temperature. The RBCs were then suspended in SAGM (150 mmol/L NaCl, 1.25 mmol/L adenine, 50 mmol/L glucose, 29 mmol/L mannitol, pH 5.6) (Fresenius Kabi Sèvres, France) to constitute the RBC unit. The RBC units arrived at our laboratory two to three days after blood collection and processing and were stored at +4 to 6° C. in a standard blood-bank refrigerator until day 42.

Samples were taken at two different storage times (D3-D8 and D38-D42) to evaluate spectral characteristics of Hb and quantify the soluble α-Hb pool in RBCs. Two additional samples were collected from sixteen RBC units, at intermediate time points during storage (D13-D17 and D24-D29; n=16). The soluble α-Hb pool is determined using the quantitative method that inventors developed to measure the amount of soluble α-Hb chains directly in the RBC lysates. This α-Hb dosing assay uses the specific character of the interaction between the α-Hb and the α-haemoglobin stabilizing protein (AHSP), the α chaperone, to trap the α-Hb present in the RBC lysates.¹⁷ They also investigated the effect of a short cryopreservation period at −80° C. for 15 days on the α-Hb pool for 4 different RBC units.

Accordingly, in a first aspect, the invention relates to a method for determining the quality of Hb during the storage period of RBC units comprising a step of measuring the value of soluble alpha-haemoglobin (α-Hb) pool in RBC units and concluding a conservation of quality of Hb during the storage of RBCs when the value of α-Hb remains stable and does not increase significantly.

In a particular embodiment, the method according to the invention comprises further the following steps:
i) evaluating the value of soluble α-Hb pool in RBC units at the beginning of storage;
ii) evaluating the value of soluble α-Hb pool in RBC units at the end of storage;
iii) comparing the values of soluble α-Hb pool measured between the beginning and the end of storage; and
iv) concluding that the quality of Hb during the storage period of RBCs is conserved during the storage period when the value of soluble α-Hb pool is maintained during the storage period of RBC units; or concluding that the quality of Hb during the storage period of RBC units is not conserved during the storage period when the value of soluble α-Hb pool is increased significantly during the storage period of RBC units.

As used herein, the term “red blood cells” (RBCs), also called as red cells, red blood corpuscles, erythroid cells or erythrocytes are the most common type of blood cell and the vertebrate's principal means of delivering oxygen to the body tissues via blood flow through the circulatory system. RBCs take up oxygen in the lungs, and release it into tissues while squeezing through the body's capillaries. The cytoplasm of RBC is rich in haemoglobin, an iron-containing biomolecule that can bind oxygen and is responsible for the red color of the cells and the blood.

As used herein, the term “RBC lysates” refers to RBCs which are lysed for example with four volumes of cold distilled water. The mixture was incubated for 30 min on ice, centrifuged at 16,000×g for 30 minutes at +4° C. and RBC lysates were recovered in the supernatant and immediately frozen at −80° C. In the context of the invention, the value of soluble α-Hb pool is evaluated in RBCs lysates.

As used herein, the term “RBC units” also called as red blood cell concentrates or red cell concentrates. RBC units refer to RBCs which are prepared from whole blood by removing the plasma fraction after centrifugation and are leuco-filtered at room temperature and then re-suspended into a SAGM solution according with European guidelines.¹⁹

As used herein, the term haemoglobin (Hb) is the iron-containing oxygen-transport metalloprotein in RBC. Hb in blood carries oxygen from the lungs or gills to the rest of the body (i.e. the tissues). There it releases the oxygen to permit aerobic respiration to provide energy to power the functions of the organism in the process called metabolism.

The normal development of RBC requires a coordinated synthesis of the Hb subunits, the α- and β-globins in the case of adult human haemoglobin (Hb A). The α- and β-globin chains are encoded by genes on different chromosomes, 16 and 11 respectively, and their expression is controlled independently. In the normal RBC, slightly more α-chains than β-chains are produced. Unlike the β-haemoglobin chains (β-Hb) which are soluble and form homologous tetramers, the free α-Hb are highly instable, and when in excess, form precipitates and act as active oxidants causing apoptosis and inefficient erythropoiesis.

As used herein, the term “α-Hb” refers to a 141 amino acid protein also called alpha-haemoglobin (HBA1 and HBA2 genes), alpha-globin chain with haem or alpha chain. α-Hb protein corresponds to GenBank accession number NP_000549. Usually, in healthy subjects, Hb A consists of four protein subunits, two subunits called α-Hb and two subunits called β-Hb.

As used herein, the terms “soluble α-Hb pool” or “free α-Hb pool” correspond to the alpha-Hb chains (or monomers) which are not bound to β-Hb in RBCs or reticulocytes but that can be linked to AHSP. Thus, free α-Hb corresponds to the relative excess of alpha-Hb chains which are not bound to the red blood cell membranes or not aggregated (inclusion bodies).

As used herein, the term “quality” refers to maintain of Hb's functions during the storage period. Typically, Hb is the iron-containing oxygen-transport metalloprotein in the RBCs. The quality in the context of the invention refers to the ability of Hb to carry oxygen from the lungs or gills to the rest of the body. Accordingly, the method according to the invention allow to identify this ability and thus whether RBCs units can be used after a storage period.

As used herein, the term “maintained” or “remains stable” refers to maintain of low value of soluble α-Hb pool during storage period. Typically, during the storage period, the value of soluble α-Hb pool is not significantly increased during the first fourteen days of storage and thus the spectrum of Hb is identical (=similar) at the beginning and the end of the storage period. Accordingly, when the value of soluble α-Hb pool remains stable or increases not significantly during the storage period, it means that the quality of Hb is conserved. In the contrary, when the value of soluble α-Hb pool is increased significantly during this storage period, it means that the quality of Hb is not conserved and thus should be withdraw from the storage.

As used herein, the term “storage” refers to a step where RBCs are conserved. Standard solutions for the storage of whole blood include citrate-phosphate-dextrose solution (CPD) and citrate-phosphate-dextrose-adenine solution (CPDA) as components of additive solutions. Citrate or other anticoagulants such as heparin, ethylenediaminetetraacetic acid (EDTA) are necessary to prevent clotting. Because blood is a living tissue that maintains metabolic functions even at refrigerated temperatures, it has been considered necessary to provide an energy source such as dextrose. Phosphate ion can be used to buffer the lactate produced from dextrose utilization. Other components of additive solutions include salts and buffers to help maintain physiological plasma pH conditions. Nucleobases such as adenine and nucleosides such as guanosine may also be added. Thus, standard solutions for the storage of RBCs include SAGM solution.

RBCs are stored in a bag, they can be affected by storage conditions and have storage lesion. As used herein, the term “storage lesion” refers to structural and functional changes to store RBCs in a storage bag.

In a particular embodiment, the storage container is a container, pouch, bag, or bottle that is constructed of a material compatible with a biological fluid, such as whole blood or a blood component and is capable of withstanding centrifugation and sterilization. Such containers are known in the art and include, e.g., for example, blood collection and satellite bags. Storage containers can be made of plasticized polyvinyl chloride, e.g., PVC plasticized with dioctylphthalate, diethylhexylphthalate, or trioctyltrimellitate. The bags may also be formed from polyolefin, polyurethane, polyester, and polycarbonate. Once transferred to the storage container, the RBC sample can be stored under aerobic or anaerobic conditions, i.e., conditions of low or no oxygen.

Typically, in the context of the invention, the blood from twenty-one healthy adult donors was collected into sterile blood bags containing citrate phosphate dextrose (CPD) as an anticoagulant, at the Etablissement Francais du Sang (EFS). These twenty-one fresh non-therapeutic bloods were maintained at a temperature between +18° C. and +24° C. for 2 to 24 hours before processing, in accordance with European guidelines¹⁹, at the EFS Preparation Unit (Rungis, France). The RBCs were isolated by removal of plasma and leukoreduction at room temperature. The RBCs were then suspended in SAGM to constitute the RBC units.

In a particular embodiment, the RBC units were received in the laboratory at days 2 to 3 after their collection and were stored at +4 to 6 ° C. in a standard blood bank refrigerator until day 42.

In a particular embodiment, the storage period refers to the period where the RBC units are conserved in a storage container. Typically, the storage period is 42 days.

In a particular embodiment, the method according to the invention is performed at days 3 to 8 (D3-D8) and days 38 to 42 (D38-D42) after collection. Two additional samples were collected from sixteen RBC units, at intermediate time points during storage (D13-D17 and D24-D29; n=16).

In a particular embodiment, the value of soluble α-Hb pool between day 8 and day 18 remains stable or does not increase significantly. In the context of the invention, the value of soluble α-Hb pool during this period is of a similar amount to that at the beginning of storage.

In another embodiment, the value of soluble α-Hb pool is increased significantly at day 42 compared to its value at the beginning of the storage.

As used herein, the term “evaluating” means determining the value of α-Hb in a biological sample.

As used herein, the term “value”, refers to the value of absorbance determined in-visible absorbance at 414 nm. Typically, the value is measured at the beginning and end of the storage period at 414 nm, wavelength which proteins other than haemoprotein were not detected.

As used herein, the value of the soluble α-Hb pool refers to the amount of the soluble α-Hb pool in RBC units. The amount of the soluble α-Hb pool in RBC units is evaluated through a specific quantitative method developed by the inventors (Vasseur C et al; Am J Hematol; 86: 199-202 (2011).

In a particular embodiment, the measurement of value of soluble α-Hb is performed according to the method described in WO2010/122160.

In a particular embodiment, the method according to the invention, wherein the evaluation of value of soluble α-Hb is performed by mass spectrometry.

In a particular embodiment, the method according to the invention, wherein the evaluation of value of soluble α-Hb is performed by affinity chromatography with glutathione-Sepharose 4B beads.

In a particular embodiment, the method according to the invention, wherein the mass spectrometry is gas-chromatography/mass spectrometry (GC/MS) or liquid chromatography-tandem mass spectrometry (LC/MS/MS).

As used herein, the term “mass spectrometry” or “MS” refers to an analytical technique to identify compounds by their mass. MS refers to methods of filtering, detecting, and measuring ions based on their mass-to-charge ratio, or “m/z”. MS technology generally includes ionizing the compounds to form charged compounds; and detecting the molecular weight of the charged compounds and calculating a mass-to-charge ratio. The compounds may be ionized and detected by any suitable means. A “mass spectrometer” generally includes an ionizer and an ion detector. In general, one or more molecules of interest are ionized, and the ions are subsequently introduced into a mass spectrographic instrument where, due to a combination of magnetic and electric fields, the ions follow a path in space that is dependent upon mass (“m”) and charge (“z”). See, e.g., U.S. Pat. No. 6,204,500, entitled “Mass Spectrometry From Surfaces;” U.S. Pat. No. 6,107,623, entitled “Methods and Apparatus for Tandem Mass Spectrometry;” U.S. Pat. No. 6,268,144, entitled “DNA Diagnostics Based On Mass Spectrometry;” U.S. Pat. No. 6,124,137, entitled “Surface-Enhanced Photolabile Attachment And Release For Desorption And Detection Of Analytes;” Wright et al., Prostate Cancer and Prostatic Diseases 1999, 2: 264-76; and Merchant and Weinberger, Electrophoresis 2000, 21; 1164-67.

As used herein, the term “gas chromatography” or “GC” refers to chromatography in which the sample mixture is vaporized and injected into a stream of carrier gas (as nitrogen or helium) moving through a column containing a stationary phase composed of a liquid or a particulate solid and is separated into its component compounds according to the affinity of the compounds for the stationary phase.

As used herein, the term “liquid chromatography” or “LC” means a process of selective retardation of one or more components of a fluid solution as the fluid uniformly percolates through a column of a finely divided substance, or through capillary passageways. The retardation results from the distribution of the components of the mixture between one or more stationary phases and the bulk fluid, (i.e., mobile phase), as this fluid moves relative to the stationary phase(s). Examples of “liquid chromatography” include reverse phase liquid chromatography (RPLC), high performance liquid chromatography (HPLC), and turbulent flow liquid chromatography (TFLC) (sometimes known as high turbulence liquid chromatography (HTLC) or high throughput liquid chromatography).

In a particular embodiment, the method according to the invention, wherein the measurement of value of soluble α-Hb is performed by enzymatic assay.

In a particular embodiment, the method according to the invention, wherein the measurement of value of soluble α-Hb is performed by ELISA.

In a particular embodiment, the method according to the invention, wherein the measurement of value of soluble α-Hb is performed by affinity chromatography with GST-AHSP-coupled to a glutathione Sepharose 4B coated to 96-well filter plates.

Immunoassay techniques and protocols are generally described in Price and Newman, “Principles and Practice of Immunoassay,” 2nd Edition, Grove's Dictionaries, 1997; and Gosling, “Immunoassays: A Practical Approach,” Oxford University Press, 2000. A variety of immunoassay techniques, including competitive and non-competitive immunoassays, can be used. See, e.g., Self et al., Curr. Opin. Biotechnol., 7:60-65 (1996). The term immunoassay encompasses techniques including, without limitation, enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (META); immunohistochemical (IHC) assays; capillary electrophoresis immunoassays (CEIA); radioimmunoassays (MA); immunoradiometric assays (IRMA); fluorescence polarization immunoassays (FPIA); and chemiluminescence assays (CL). If desired, such immunoassays can be automated Immunoassays can also be used in conjunction with laser induced fluorescence. See, e.g., Schmalzing et al., Electrophoresis, 18:2184-93 (1997); Bao, J. Chromatogr. B. Biomed. Sci., 699:463-80 (1997). Liposome immunoassays, such as flow-injection liposome immunoassays and liposome immunosensors, are also suitable for use in the present invention. See, e.g., Rongen et al., J. Immunol. Methods, 204:105-133 (1997). In addition, nephelometry assays, in which the formation of protein/antibody complexes results in increased light scatter that is converted to a peak rate signal as a function of the marker concentration, are suitable for use in the methods of the present invention. Nephelometry assays are commercially available from Beckman Coulter (Brea, Calif; Kit #449430) and can be performed using a Behring Nephelometer Analyzer (Fink et al., J. Clin. Chem. Clin. Biochem., 27:261-276 (1989)).

Specific immunological binding of the antibody to proteins can be detected directly or indirectly. Direct labels include fluorescent or luminescent tags, metals, dyes, radionuclides, and the like, attached to the antibody. A chemiluminescence assay using a chemiluminescent antibody specific for the protein is suitable for sensitive, non-radioactive detection of protein levels. An antibody labeled with fluorochrome is also suitable. Examples of fluorochromes include, without limitation, DAPI, fluorescein, Hoechst 33258, R-phycocyanin, B-phycoerythrin, R-phycoerythrin, rhodamine, Texas red, and lissamine Indirect labels include various enzymes well known in the art, such as horseradish peroxidase (HRP), alkaline phosphatase (AP), β-galactosidase, urease, and the like. A horseradish-peroxidase detection system can be used, for example, with the chromogenic substrate tetramethylbenzidine (TMB), which yields a soluble product in the presence of hydrogen peroxide that is detectable at 450 nm. An alkaline phosphatase detection system can be used with the chromogenic substrate p-nitrophenyl phosphate, for example, which yields a soluble product readily detectable at 405 nm. Similarly, a β-galactosidase detection system can be used with the chromogenic substrate o-nitrophenyl-β-D-galactopyranoside (ONPG), which yields a soluble product detectable at 410 nm. An urease detection system can be used with a substrate such as urea-bromocresol purple (Sigma Immunochemicals; St. Louis, Mo.).

A signal from the direct or indirect label can be analyzed, for example, using a spectrophotometer to detect color from a chromogenic substrate; a radiation counter to detect radiation such as a gamma counter for detection of I¹²⁵; or a fluorometer to detect fluorescence in the presence of light of a certain wavelength. For detection of enzyme-linked antibodies, a quantitative analysis can be made using a spectrophotometer such as an EMax® Microplate Reader (Molecular Devices; Menlo Park, Calif.) in accordance with the manufacturer's instructions. If desired, the assays of the present invention can be automated or performed robotically, and the signal from multiple samples can be detected simultaneously.

The antibodies can be immobilized onto a variety of solid supports, such as magnetic or chromatographic matrix particles, the surface of an assay plate (e.g., microtiter wells), pieces of a solid substrate material or membrane (e.g., plastic, nylon, paper), and the like. An assay strip can be prepared by coating the antibody or a plurality of antibodies in an array on a solid support. This strip can then be dipped into the test sample and processed quickly through washes and detection steps to generate a measurable signal, such as a colored spot.

In a particular embodiment, the method according to the invention, wherein the measurement of value of soluble α-Hb is performed with a method using the α-chaperone to capture specifically the available α-Hb present in RBC lysates.

In a particular embodiment, the method according to the invention, wherein α-chaperone is AHSP. Typically, the measurement of the α-Hb pool required upstream the preparation of recombinant AHSP. Recombinant AHSP was produced as a fusion protein with glutathione S-transferase (GST-AHSP) in E.coli and purified by affinity chromatography using glutathione-Sepharose 4B beads (GE Healthcare, Lifescience, Uppsala, Sweden) as previously described.²¹ The purified GST-AHSP was preserved at −80° C. in phosphate buffered saline containing 1% bovine serum albumin and 10% glycerol.

### Uses of the Method According to the Invention

The method as described above is suitable to be used in a transfusion.

The method as described above is suitable to be used to determine if the RBC units is suitable for transfusion.

According to the invention, it is concluded that the RBC units stored are suitable to be used in a transfusion when the conservation of quality of Hb during the storage of RBCs is determined according to the method as described above.

Accordingly, in a second aspect, the present invention relates to a method of determining the suitability of RBC units for transfusion.

The invention relates to a method of determining the suitability of RBC units for transfusion comprising steps of i) determining the quality of Hb during the storage period of RBCs comprising a step of measuring the value of soluble α-Hb pool in RBC units; ii) concluding a conservation of quality of Hb during the storage of RBCs when the value of α-Hb remains stable or is not significantly increased and iii) concluding that the RBC units stored are suitable to be used in a transfusion.

The method according to the invention comprising a step of i) determining the quality of Hb during the storage period of RBCs comprising a step of measuring the value of soluble α-Hb pool in RBC units; ii) concluding a conservation of quality of Hb during the storage of RBCs when the value of α-Hb pool remains stable or is not significantly increased during the first fourteen days of storage and iii) concluding that the RBC units stored are suitable to be used in a transfusion.

In a particular embodiment, the method of determining the suitability of RBC units for transfusion comprises further the following steps:
i) evaluating the value of soluble α-Hb pool in RBC lysates at the beginning of storage;
ii) evaluating the value of soluble α-Hb pool in RBC lysates at the end of storage;
iii) comparing the values of soluble α-Hb pool measured between the beginning and the end of storage; and
iv) concluding that the quality of Hb during the storage period of RBCs is conserved during the storage period when the value of soluble α-Hb pool remains stable or not significantly increases during the storage period of RBCs; or concluding that the quality of Hb during the storage period of RBCs is not conserved during the storage period when the value of soluble α-Hb pool is significantly increased during the storage period of RBCs;
v) concluding that RBC units are suitable for transfusion when the value of soluble α-Hb pool remains stable or not significantly increases during the storage period of RBCs.

In a particular embodiment, the value of soluble α-Hb pool in RBC units is measured at days 8 to 18 (D8-D18) of the storage period of RBCs.

In a particular embodiment, when the value of soluble α-Hb pool between day 8 and day 18 remains stable, the transfusion can be performed. In the context of the invention, the value of soluble α-Hb pool during this period is of a similar amount to that at the beginning of storage and thus the transfusion can be performed.

In another embodiment, when the value of soluble α-Hb pool is increased significantly (e.g. at day 42 compared to its value at the beginning of the storage), the transfusion cannot be performed.

As used herein, the term “transfusion” refers to an event where blood is removed from one individual (donor), animal, or human, and transfused to another in need (recipient).

As used herein, the term “donor” refers to a human or animal, donating blood.

In a particular embodiment, the donor is a healthy human.

In another embodiment, the donor is a healthy animal.

As used herein, the term “recipient” refers to a corresponding human or animal receiving blood.

In a particular the recipient is a human suffering from sickle cell disease (SCD) and/or β-thalassemia.

In a further embodiment, the recipient is susceptible to have Hb H (β4 tetramer) disease with only one functional alpha gene.

In another embodiment, the recipient is an elderly person.

In another embodiment, the recipient is pregnant.

In another embodiment, the recipient is a human susceptible to have a surgery.

Typically, the transfusion is initiated when a venous device is in both donor and recipient, e.g., needle, PRN adapter, catheter in a vein, and is connected to the system. However, in some embodiments, hose clamps or other devices that block blood flow are used on the tubing to prevent the transfusion from starting before desired. Transfusion would then begin when the clamp or block is removed. Typically, transfusion is performed by following the instructions provided in “The Guide to the preparation, use and quality assurance of blood components is published by the European Directorate for the Quality of Medicines & HealthCare of the Council of Europe (EDQM)”.

As used herein, the term “venous device” refers to a sterile surgical needle standard for blood transfusions. Such needles are well known in the art.

As used herein, the term “tubing” refers to medical tubing of the type generally accepted for use in doing blood transfusions. Such tubing is readily available. In the practice of the present invention, a first transfusion venous device attaches to one end of the tubing and a second transfusion venous device is attached to the second end with the first venous device inserted in the vein of the donor and the second venous device inserted in the vein of the recipient.

As used herein, the term “blood counter” refers to a device which can determine how much flowing blood is passing by a given point in the system from the donor to the recipient. It can do that by direct measure, mechanical or digital, of the blood flowing through the blood counter.

### Kit

In a third aspect, the invention relates to a kit for use in the method of the invention as described here above, said kit comprising, as separate elements: a solid support, and an α-Hb-specific binding partner.

In a particular embodiment, said α-Hb-specific binding partner is AHSP.

In a particular embodiment, ASHP is fused to GST.

In another embodiment, said α-Hb-specific binding partner is coated directly or indirectly to a solid support, said solid support comprising a protein binding surface such as a microtiter plat, well filter plates coated with affinity resin (GST MultiTrap 4B), a colloid metal particle, an iron oxide particle, a latex particle or a polymeric bead or a column such as a GST microspin column or a nickel bead column or any affinity support that recognizes specifically the Tag or fusion moiety.

In a particular embodiment, said solid support is a GST microspin column or GST MultiTrap 4B. In a particular preferred embodiment, GST-AHSP is fixed on GST microspin column or to 96-well filter plates coated with glutathione Sepharose 4B.

The kit may also contain optional additional components for performing the method of the invention. Such optional components are for example containers, mixers, buffers, instructions for assay performance, labels, supports, and reagents necessary to elution.

The invention will be further illustrated by the following figures and examples. However, these examples and figures should not be interpreted in any way as limiting the scope of the present invention.

### FIGURES

FIG. 1: UV-visible absorbance spectra of lysates in stored blood units. The UV-visible absorbance spectra for soluble Hb in cytosols were obtained in RBC lysates from blood units from 250 to 700 nm, at the beginning (D3-D8, black line) and end (D38-D42, black dotted line) of the 42-day storage period. The spectra show a visible absorbance band typical of haem with a Soret band at 415 nm; the ratio of absorbance intensities at the Soret band and the UV peak at 280 nm were about 3.70±0.06 for D3-D8 and 3.79±0.04 for D38-D42. The spectra obtained were similar to that of native oxygenated Hb¹⁶ and in the ferrous form required for oxygen transport. So, the characteristics of soluble Hb were correctly conserved during a long-refrigerated storage and do not lead to significant methaemoglobin formation (data not shown), the latter would be at the origin of denatured Hb bound to the membrane. Representative values for five lysates are shown. Absorbance spectra were measured with an Eon™ microplate spectrophotometer.

FIG. 2: Detection and follow-up of the soluble α-Hb pool in RBC lysates. (A) Changes in the α-Hb pool at four different time points during storage. Measurements were made on lysates from sixteen leukoreduced RBC units, stored at +4 to 6° C. for 42 days, on D3-D8, D13-D17, D24-D29 and D38-D42. Each symbol represents a different RBC unit and each α-Hb value is the mean of two measurements. Statistical analyses were performed with Friedman's test followed by Dunn's multiple comparisons test. *p<0.05, ***p<0.001. (B) Effect of a freezing/thawing procedure on the α-Hb pool. Measurements were made on four RBC lysates from freshly prepared leukoreduced RBC units stored in SAGM for 3 to 8 days (D3-D8) and from the same units after freezing at D3-D8 and thawing 15 days after. Each symbol represents a different RBC unit; each α-Hb value is the mean of two measurements.

FIG. 3: Effect of storage time on the soluble α-Hb pool in RBC units. The α-Hb pool was detected in RBC lysates from leukoreduced RBC units (n=21) stored at +4 to 6° C. Measurements were made at the beginning and end of storage (D3-D8 versus D38-D42). Each α-Hb value is the mean of two measurements. Statistical analyses were performed with Wilcoxon matched-pairs signed-rank test. *p<0.05, ***p<0.001, ****p<0.0001. Results are shown as box-and-whisker plots with individual values indicated as dots; horizontal bars indicate the median.

### EXAMPLE

Material & Methods

RBC Units, Storage and Sampling

This research was performed in accordance with the Helsinki Declaration and was approved by our institutional ethics committee (CPP no. 11-047). Blood from twenty-one healthy adult donors was collected into sterile blood bags containing citrate phosphate dextrose (CPD) as an anticoagulant, at the Etablissement Français du Sang (EFS). These fresh samples were maintained at a temperature of +18° C. to +24° C. for 2 to 24 hours before processing, in accordance with European guidelines¹⁹, at EFS Preparation Unit (Rungis, France). RBCs were isolated by plasma removal and leukoreduction at room temperature. The RBCs were then suspended in SAGM to constitute the RBC unit.

The RBC units arrived at our laboratory two to three days after blood collection and processing and were stored at +4 to 6° C. in a standard blood-bank refrigerator until day 42. We checked that none of the selected units displayed the sickle-cell trait but a deletion in one or two of the four α-globin genes cannot be totally excluded. Two samples were removed aseptically from the RBC units during the storage period with sampling couplers (Fenwal Inc., Lake Zurich, IL, USA), 3 to 8 days after collection (D3-D8; n=21) and 38 to 42 days after collection (D38-D42; n=21); two additional samples were collected from sixteen RBC units, at intermediate time points during storage (D13-D17 and D24-D29; n=16). Samples were also collected from four RBC units at D3-D8 for an alternative cryopreservation/thawing process involving the use of 57% glycerol (SpA Lab. Farmacologico, Bergamo, Italy) as a cryoprotective agent, as previously described²⁰. The glycerol-treated RBCs were immediately frozen and stored for 15 days at −80° C. They were then thawed in a +40° C. water bath, processed for automatic deglycerolisation on a Cobe 2991 machine (Terumo BCT, Inc, Lakewood, CO, USA) and used for investigations.

Preparation of RBC Lysates and Preservative Solutions

We centrifuged aliquots of RBC units at 2,880×g for 10 min at +4° C., to separate the RBCs from the preservative solution. The preservative solution was centrifuged again at 2,880×g for 10 min at +4° C., to eliminate all traces of RBCs. The various RBC fractions and preservative solutions were then frozen at −80° C. until use. RBCs were thawed gently and lysed with four volumes of cold distilled water. The mixture was incubated for 30 min on ice, centrifuged at 16,000×g for 30 min at +4° C., and RBC lysates were recovered in the supernatant and immediately frozen at −80° C.

Absorbance Spectra and Determination of Hb Concentration

Hb concentrations were determined with an Eon™ microplate spectrophotometer (BioTek Instruments Inc, Winooski, VE, USA) in the Soret band at 415 nm, for all RBC lysates obtained at the beginning (D3-D8) and end (D38-D42) of the storage period. For five RBC units, UV-visible absorbance spectra were obtained for wavelengths of 250 to 700 nm at the beginning and end of the storage period. For preservative solutions, Hb concentrations were measured by determining absorbance at 415 nm with an extinction coefficient of 125 mM⁻¹cm⁻¹, and at 540 nm by the cyanmethaemoglobin method (Drabkin's method), with an extinction coefficient of 11 mM⁻¹cm⁻¹. All the Hb concentrations are expressed on a haem basis.

Preparation of GST-AHSP Protein

Assessments of the α-Hb pool required the upstream preparation of recombinant AHSP. Recombinant AHSP was produced as a fusion protein with glutathione S-transferase (GST-AHSP) in E.coli and purified by affinity chromatography with glutathione-Sepharose 4B beads (GE Healthcare, Lifescience, Uppsala, Sweden) as previously described.²¹ The purified GST-AHSP was stored at −80° C. in phosphate buffered saline (150 mM NaCl, 10 mM Na₂HPO₄, pH 7.4) containing 1% bovine serum albumin and 10% glycerol.

Assessment of the in Vitro Soluble α-Hb pool in RBC Lysates

The α-Hb assay makes use of the specific nature of the interaction between the α-Hb and the AHSP, the a chaperone, to trap the α-Hb present in RBC lysates.¹⁷ It was performed as previously described.²² Briefly, 500 μL of RBC lysate were applied to 96-well filter plates (GST MultiTrap 4B, GE Healthcare, Lifescience, Uppsala, Sweden) coated with the GST-AHSP—coupled to glutathione Sepharose 4B. The plates were washed with phosphate buffer saline and the bound proteins (GST-AHSP and GST-AHSP/α-Hb complexes) were eluted with 10 mM reduced glutathione in 50 mM Tris-HCl buffer at pH 8.0. The quantity of α-Hb in the eluted fraction was determined by spectrophotometry at 414 nm (on a haem basis) with an Eon™ microplate reader and the data were analysed with Gen5 software. The best analytical wavelength for α-Hb pool detection was 414 nm, at which proteins other than haemoprotein were not detected; in parallel, the total Hb concentration of the RBC lysates was determined. The α-Hb value was expressed in ppm, equivalent to ng of α-Hb per mg total Hb subunits per mL of RBC lysate, to take RBC Hb concentration into account. The α-Hb pool values reported are the means of two independent measurements.

Statistical Analysis

Quantitative variables are expressed as arithmetic mean±standard deviation (SD). Data were analyzed with Prism 6.0 software (GraphPad, La Jolla, CA). We performed non-parametric tests, Friedman tests followed by Dunn's multiple comparisons test, Wilcoxon matched-pairs signed-rank tests and Mann Whitney tests. P values <0.05 were considered statistically significant.

Results

Hb in Stored RBC Lysates and Supernatants

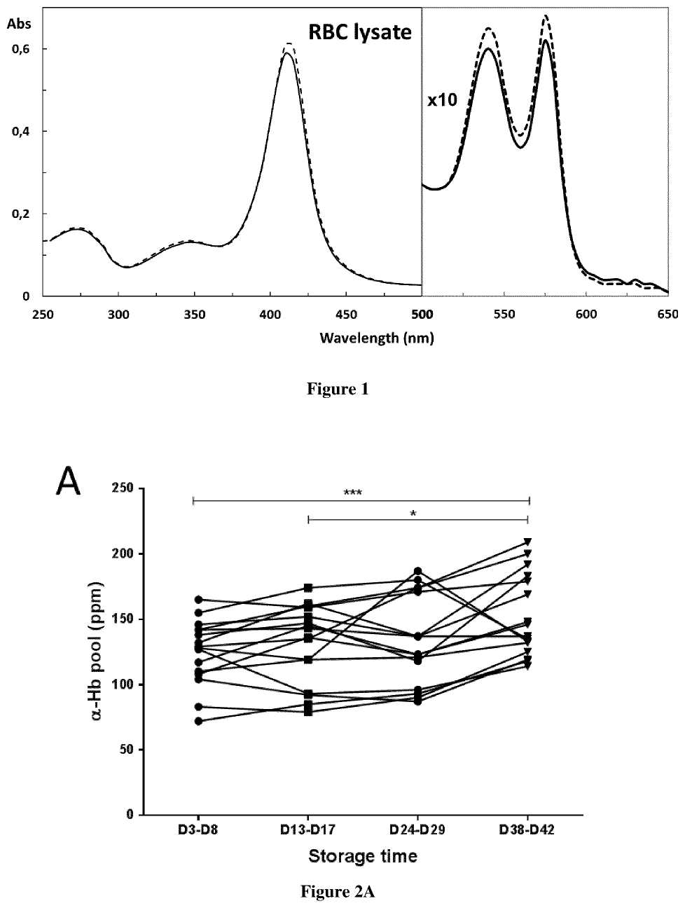

We evaluated the UV and visible absorbance spectra of RBC lysates from RBC units at the beginning (D3-D8) and end (D38-D42) of the period of storage (42 days). All RBC lysate spectra had a visible absorbance band typical of haem, with a Soret band at 415 nm (FIG. 1). The ratio of absorbance intensities at the Soret band and the UV peak at 280 nm was about 3.70±0.06 for D3-D8 and 3.79±0.04 for D38-D42 (n=5). These ratios are not significantly different from that for oxygenated native Hb A.¹⁶ We determined the Hb concentrations of lysates from the twenty-one RBC units. No significant difference was observed between the beginning (D3-D8; 3.79±0.67 mM on a haem basis) and end (D38-D42; 3.85±0.65 mM) of storage (FIG. 1). Hb was also determined in the preservative solutions (i.e. supernatants) of the twenty-one RBC units analysed (data not shown), by two spectroscopic methods, at 540 nm after cyanmet Hb transformation and directly at 415 nm. The supernatant Hb concentrations based on absorbance at 540 and 415 nm were similar, at 0.020±0.012 mM at D3-D8 versus 0.049±0.022 mM at D38-D42, corresponding to a small but significant difference between these time points (p=0.0019).

Soluble α-Hb Pool in the Lysates of Stored RBC Units

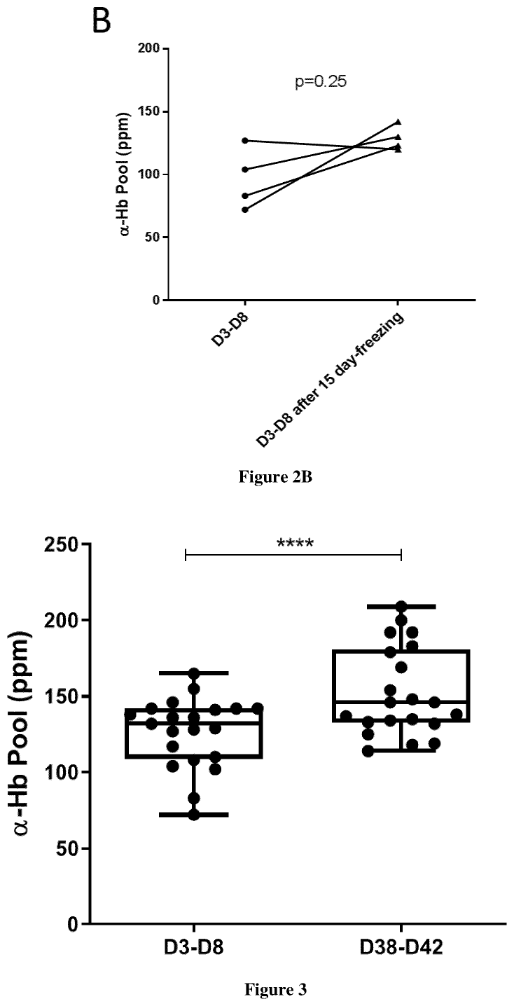

We investigated the presence of a soluble α-Hb pool in lysates from 16 RBC units after storage at +4° C. to +6° C. for various times (FIG. 2A). The “soluble α-Hb pool” corresponds to the α-Hb not bound to β-Hb in RBCs but that can be linked to AHSP.¹⁷ An α-Hb pool was detected at D3-D8, at the beginning of storage, just after the preparation of RBC units from whole blood. At this time, values ranged from 72 to 165 ppm, with a mean value of 126±23 ppm (equivalent to a mean of 7.71 μg of α-Hb bound to resin-coupled GST-AHSP protein). A dispersion of the values of different RBC units was also observed (interquartile range=33). At the two intermediate times (D13-D17 and D24-D29; n=16), mean α-Hb pool amounts were 131±30 ppm and 134±34 ppm, respectively. At the end of storage, α-Hb pool was between 114 and 209 ppm, with a mean value of 152±29 ppm (corresponding to a mean of 8.95 μg α-Hb bound to GST-AHSP). A more pronounced dispersion of α-Hb values between RBC units was observed at D38-D42 (interquartile range=48.5); the values obtained at D38-D42 were also significantly higher than those obtained at D3-D8 (n=21, p <0.0001) (FIG. 3).

The freezing of RBC units with rare blood phenotypes may be required for transfusion in specific populations of recipients, such as the sickle-cell disease patients studied by our team. We therefore also investigated the effect of cryopreservation on the α-Hb pool of RBC units. We assessed the effect of a short period of freezing on soluble α-Hb pool amount in four RBC units, by comparing samples selected at D3-D8 before (97±24 ppm) and after freezing for 15 days at −80° C. (129±10 ppm). The α-Hb pool of these samples were slightly, but not significantly higher (n=4; p=0.25) after freezing for 15 days (FIG. 2B).

Discussion

Previous studies by our team²⁰ on the same blood units stored for 42 days showed no significant changes in RBC volume, osmotic resistance, or mean corpuscular Hb concentration (MCHC) over time, providing evidence that conventional storage of RBC units did not modify RBC rheology. By contrast, and as expected, the pH of the unit supernatants decreased rapidly^{6, 20}.

The presence of isolated Hb subunits during storage of RBCs units has never previously been studied. An α-Hb pool is detected in blood units from the beginning of storage (D3-D8) at temperatures from +4 ° C. to +6 ° C., increasing over the 42-day storage period. The term “soluble α-Hb pool” corresponds to the α-Hb not bound to β-Hb that can be linked to AHSP in RBCs. The detection of such an α-Hb pool in blood units is not surprising, given that the presence of α-Hb has already been reported in RBC lysates obtained from healthy volunteers with a normal Hb phenotype²²; in that context, the mean value was 81±15 ppm, with a lesser degree of dispersion (54-115 ppm; interquartile range 21).

The differences observed in α-Hb pool values between that of RBC units and that of freshly prepared RBCs from healthy volunteers can be explained by the difference in storage temperatures after collection, in the use of different anticoagulants, or in the sample preparation. In fact, for the preparation of RBC units, whole peripheral blood is collected into CPD-anticoagulated bags and kept at room temperature for 2-20 hours before processing¹⁹. Leukoreduction is then performed by filtration before the transfer of the RBCs to a bag containing SAGM; all these steps are performed at room temperature, taking a mean time of 10-24 hours, before storage at temperatures from +4 ° C. to +6 ° C. in blood banks. By contrast, the preparation of fresh RBCs drawn from volunteers was processed at +4 ° C. within two hours of collection on EDTA and no leukoreduction step was carried out¹⁷. Furthermore, we have observed that α-Hb pool values tend to increase when the whole blood sample is stored for a period of time at room temperature (unpublished observations). It has been also reported that temperature influences the kinetics of dissociation of the αβ dimers into α and β monomers: an increase in temperature from +7 ° C. to +37 ° C. resulted in a 50-fold increase in the dissociation rate constant. All these data indicate that an increase in temperature, along with the duration of the procedure in processing the RBC unit, can have an impact on the α-Hb pool values detected and may explain the increase in the α-Hb pool in blood units.

An excess of α-Hb chains that, by precipitating on the RBC membrane and acting as active oxidants, leads to oxidant damage has previously been reported^{24,25}. In our team, we initially detected the α-Hb pool in RBCs drawn from pathological β-thalassemia blood samples^{17,22}. In this Hb disorder, an imbalance in the biosynthesis of globin chains lead to an excess of α-chains. Precipitation of α-chains, and oxidative damage in erythroid precursors and RBCs, resulting in inefficient erythropoiesis, have all been observed. In the most severe forms of β-thalassemia, the α-Hb pool values are higher than 1,000 ppm (very high in comparison to the α-Hb pool observed in RBC units) and this correlates well with the clinical severity of the disease. Here, the detected α-Hb pool value is negligible compared to the amount of functional Hb in RBC units and would have had almost no impact on the quality of the stored RBC units. Furthermore, most blood units are transfused to patients between D8 and D18, and the α-Hb pool remains practically stable within this timeframe (FIG. 2A), thus supporting the view that the values of α-Hb would not affect the choice of RBC units to be transfused.

The wide dispersion of values from different RBC units observed throughout the storage period, but also increasing towards the end (interquartile range 33 at D3-D8 vs 48.5 at D38-D42), may reflect the well-known variability between different blood bags obtained from the same donor¹⁴. The units were verified for the lack of the sickle-cell trait but were not genotyped for globin genes. In a previous study, out of 50 healthy volunteers genotyped for globin genes, 20% had an abnormal α-globin genotype and α-Hb pool values lower than those observed in normal α-globin subjects²⁶. This could be due to the presence of an α-thalassemia mutation^{17, 22}. Thus, it would be of particular interest to know more about the α-globin genotype of those two RBC units with α-Hb values lower than the average of the other units tested (FIG. 2A; see at D3-D8).

It is important to remember that the use of cryopreserved blood units with rare phenotypes can be required for transfusion in certain circumstances, particularly for sickle-cell anaemia (SCA) patients in painful acute crisis or those experiencing severe haemolytic episodes²⁰. The results we obtained before and after freezing some blood units clearly showed that the α-Hb pool increased only slightly (FIG. 2B). Thus, such a freezing/thawing procedure required to transfuse previously cryopreserved rare blood units does not significantly modify the soluble α-Hb pool. Finally, as a control, we verified the impact of storage time on Hb functionality in stored RBCs (FIG. 1) with spectra evaluated for soluble Hb in cytosols of RBCs from blood units. The characteristics of soluble Hb were correctly preserved during a lengthy period of refrigerated storage, as shown for other RBC parameters in previous rheological studies by our team^{14, 20.}

### Conclusions

This study evaluated the Hb spectra and the presence of a soluble α-Hb pool in the RBC units throughout the 42-day storage period, to determine the impact of the storage time on quality of Hb.

Considering the conservation of RBC units, we can conclude that a higher storage temperature leads to an increase of α-Hb pool value. In conclusion, this study shows in RBC units no modifications of Hb absorbance spectra depending on the storage time indicating that the quality of Hb during the storage period is maintained.

We also show for the first time the presence of a soluble α-Hb pool in RBC units with a great variability from one RBC unit to another as well as the significant increase of this pool after a storage period of 42 days.

In conclusion, inventors show here, for the first time, the presence of a soluble α-Hb pool in RBC units, with a high variability between RBC units and a significant increase in this pool after storage for 42 days, although the final quantity of the α-Hb pool remained relatively small.

The authors demonstrate here that the increase of soluble α-Hb pool in RBCs units observed at intermediate time D13-D17 was not significantly higher than that obtained at beginning storage, important result since the most RBC units are used for transfusion between day 8 and day 18. Thus, α-Hb pool evaluation can be used in the future as a new supplementary quantitative parameter for the follow-up of the quality of RBC units for transfusion. This may improve the selection of particular blood units for the transfusion of specific populations of recipients, such as sickle-cell disease patients, who are highly dependent to the quality of the blood products they receive during transfusion.

### REFERENCES

Throughout this application, various references describe the state of the art to which this invention pertains. The disclosures of these references are hereby incorporated by reference into the present disclosure.
1. Bosman G J, Willekens F L, Werre J M. Erythrocyte aging: a more than superficial resemblance to apoptosis? Cell Physiol Biochem 2005; 16:1-8.
2. Werre J M, Willekens F L, Bosch F H, et al. The red cell revisited—matters of life and death. Cell Mol Biol 2004; 50(2):139-45. Review.
3. Devitt A, Pierce S, Oldreive C, et al. CD14-dependent clearance of apoptotic cells by human macrophages: the role of phosphatidylserine. Cell Death Differ 2003; 10:371-82.
4. Kuypers F A, de Jong K. The role of phosphatidylserine in recognition and removal of erythrocytes. Cell Mol Biol 2004; 50:147-58.
5. Pantaleo A, Giribaldi G, Mannu F et al. Naturally occurring anti-band 3 antibodies and red blood cell removal under physiological and pathological conditions. Autoimmun Rev 2008; 7:457-62.
6. D'Alessandro A, Liumbruno G, Grazzini G, Zolla L. Red blood cell storage: the story so far. Blood Transfus 2010; 8:82-8.
7. Yoshida T, Prudent M, D'Alessandro A. Red blood cell storage lesion: causes and potential clinical consequences. Blood Transfus 2019; 17(1):27-52.
8. Mollison P L, Young I M. In vivo survival in the human subject of transfused erythrocytes after storage in various preservative solutions. Exp Physiol 1942; 31:359-92.
9. Hess J R. An update of solutions for red cell storage. Vox Sang 2006; 91:13-9.
10. Bosman G J, Kay M M. Erythrocyte aging: a comparison of model systems for simulating cellular aging in vitro. Blood Cells 1988; 14:19-46.
11. Luten M, Roerdinkholder-Stoelwinder B, Bos H J, et al. Survival of the fittest?—Survival of stored red blood cells after transfusion. Cell Mol Biol 2004; 50:197-203.
12. Bosman G J, Werre J M, Willekens F L, et al. Erythrocyte ageing in vivo and in vitro: structural aspects and implications for transfusion. Transfus Med 2008; 18:335-47.
13. Hod E A, Spitalnik S L. Stored red blood cell transfusions Iron, inflammation, immunity, and infection. Transfus Clin Biol 2012; 19:84-9. Review.
14. Chadebech P, Bodivit G, Razazi K, et al. Red blood cells for transfusion in patients with sepsis: respective roles of unit age and exposure to recipient plasma. Transfusion 2017; 57:1898-1904.
15. Lang K S, Lang P A, Bauer C, et al. Mechanisms of suicidal erythrocyte death. Cell Physiol Biochem 2005; 15:195-202.
16. Antonini E, Brunori M. Haemoglobin and myoglobin in their reactions with ligands. North-Holland, Amsterdam, 1971 (U.S. distributor, Elsevier, New York). Frontiers of Biology, vol. 21.
17. Vasseur C, Pissard S, Domingues-Hamdi E, et al. Evaluation of the Free α-Haemoglobin Pool in Red Blood Cells: a New Test Providing a Scale of β-Thalassemia Severity. Am J Hematol 2011; 86:199-202.
18. Shaeffer J R. Evidence for soluble alpha-chains as intermediates in haemoglobin synthesis in the rabbit reticulocyte. Biochem Biophys Res Commun 1967; 28:647-52.
19. Council of Europe. Guide to the preparation, use and quality assurance of blood components. Recommendation n° R(95) 15 on the preparation, use and quality assurance of blood components. 14th ed. Strasbourg: Council of Europe Publishing; 2008.
20. Chadebech P, de Ménorval M A, Bodivit G, et al. Evidence of benefits from using fresh and cryopreserved blood to transfuse patients with acute sickle cell disease. Transfusion 2016; 56:1730-8.
21. Baudin-Creuza V, Vasseur-Godbillon C, Pato C, et al. Transfer of human alpha- to beta-haemoglobin via its chaperone protein: evidence for a new state. J Biol Chem 2004; 279:36530-3.
22. Vasseur C, Domingues-Hamdi E, Ledudal K, et al. Red blood cells free α-haemoglobin pool: a biomarker to monitor the β-thalassemia intermedia variability. The ALPHAPOOL study. Br J Haematol 2017; 179:142-53.
23. Bennett-Guerrero E, Veldman T H, Doctor A, et al. Evolution of adverse changes in stored RBCs. Proc Natl Acad Sci USA 2007; 104:17063-8.
24. Scott M D, van den Berg J J M, Repka T, et al. Effect of excess α-hemoglobin chains on cellular and membrane oxidation in model β-thalassemic erythrocytes. J. Clin Invest. 1993; 91: 1706-12.
25. Scott M D, Rouyer-Fessard P, Lubin B H, Beuzard Y. Entrapment of purified alpha-hemoglobin chains in normal erythrocytes. A model for beta thalassemia. J Biol Chem 1990; 265: 17953-9.
26. Vasseur C, Galactéros F, Baudin-Creuza V. α-Haemoglobin pool measurement: a useful biomarker for evaluation of β-thalassaemia intermedia?—response to Huang and Li. Br J Haematol. 2018; 183: 671-3.

1. A method for determining the quality of haemoglobin (Hb) during a storage period of red blood cell (RBC) units comprising
measuring, at least at the beginning and end of the storage period, the value of a soluble alpha-haemoglobin (α-Hb) pool in the RBC units and
determining that the quality of Hb is conserved during the storage period when the value of the soluble α-Hb pool remains stable.

2. The method according to claim 1 further comprising:
i) evaluating the value of the soluble α-Hb pool in RBC lysates at the beginning of storage;
ii) evaluating the value of the soluble α-Hb pool in RBC lysates at the end of storage;
iii) and determining that the quality of Hb is conserved during the storage period when the value of soluble α-Hb pool is maintained during the storage period; or determining that the quality of Hb during the storage period is not conserved during the storage period when the value of soluble α-Hb pool is increased during the storage period.

3. The method according to claim 1, wherein the method is performed at days 3 to 8 (D3-D8) and days 38 to 42 (D38-D42) after collection.

4. The method according to claim 1, wherein the value of the soluble α-Hb pool is measured by mass spectrometry.

5. The method according to claim 4, wherein the mass spectrometry is gas-chromatography/mass spectrometry (GC/MS) or liquid chromatography-tandem mass spectrometry (LC/MS/MS).

6. The method according to claim 1, wherein the value of the soluble α-Hb pool is measured by ELISA.

7. The method according to claim 1, wherein the value of soluble α-Hb pool is measured by affinity chromatography.

8. The method according to claim 7, wherein the affinity chromatography is performed with glutathione S-transferase-α-haemoglobin stabilizing protein (GST-AHSP)-coupled to a glutathione Sepharose 4B coated to 96-well filter plates.

9. The method according to claim 1, wherein said method determines whether the RBC units are suitable for transfusion.

10. A method of determining the suitability of RBC units for transfusion comprising i) measuring, at least at the beginning and the end of a storage period of the RBC units, the value of a soluble α-Hb pool in the RBC units; ii) determining that the stored RBC units are suitable to be used in a transfusion when the value of α-Hb remains stable or is not increased during the storage period.

11. The method according to claim 10, wherein the value of the soluble α-Hb pool in the RBC units is measured at days 3 to 8 (D3-D8) of the storage period.

12. The method according to claim 11, wherein the stored RBC units are suitable to be used in a transfusion when the value of the soluble α-Hb pool between day 8 and day 18 of the storage period remains stable or is not increased.

13. The method according to claim 10, wherein the stored RBC units are not suitable to be used in a transfusion when the value of the soluble α-Hb pool is increased during the storage period.

14. A kit for use in the method according to claim 1, comprising as separate components: a solid support, and an α-Hb-specific binding partner.
